(19) United States
(12) Patent Application Publication
(10) Pub. No.: US 2014/0058333 A1
(43) Pub. Date: Feb. 27, 2014

(54) MEDICATED MODULE WITH AUTOMATIC RESERVOIR ENGAGEMENT AND LOCK MECHANISM

(71) Applicants: John David Cross, Northhamptonshire (GB); Michael Bainton, Warwickshire (GB); Garen Kouyoumjian, Warwickshire (GB); David Richard Mercer, Warwickshire (GB); Malcolm Stanley Boyd, Warwickshire (GB)

(72) Inventors: John David Cross, Northhamptonshire (GB); Michael Bainton, Warwickshire (GB); Garen Kouyoumjian, Warwickshire (GB); David Richard Mercer, Warwickshire (GB); Malcolm Stanley Boyd, Warwickshire (GB)

(73) Assignee: SANOFI-AVENTIS DEUTSCHLAND GMBH, Frankfurt am Main (DE)

(21) Appl. No.: 14/110,548

(22) PCT Filed: Apr. 19, 2012
(86) PCT No.: PCT/EP2012/057150

§ 371 Date: Oct. 8, 2013

(30) Foreign Application Priority Data
Apr. 21, 2011 (EP) 11163364.0

(51) Int. Cl. A61M 5/24; A61M 5/00; A61M 5/32

(52) U.S. Cl. CPC A61M 5/2448; A61M 5/326; A61M 5/002
USPC 604/198

(57) ABSTRACT

A module for an injection system to co-deliver at least two medicaments is disclosed where a primary delivery device containing a primary medicament accepts a module containing a single dose of a secondary medicament and where both medicaments are delivered through a hollow needle. The module does not require the user to manually engage a reservoir containing the secondary medicament. Instead, a biasing member automatically activates the reservoir when the needle guard is retracted. The needle guard prevents accidental needle sticks before and after an injection, and locks after dose delivery. Restraining features are present on the module to prevent the needle guard from moving relative to the device, in a trigger locked position.

### CROSS REFERENCE TO RELATED APPLICATIONS

The present application is a U.S. National Phase Application pursuant to 35 U.S.C. §371 of International Application No. PCT/EP2012/057150 filed Apr. 19, 2012, which claims priority to European Patent Application No. 11163364.0 filed Apr. 21, 2011. The entire disclosure contents of these applications are herewith incorporated by reference into the present application.

### TECHNICAL FIELD

This invention relates medical devices and methods of delivering at least two drug agents from separate reservoirs using devices having only a single dose setting mechanism and a single dispense interface. This invention also relates to the secondary packaging in which the medical devices are stored and transported to a user. A single delivery procedure initiated by the user causes a non-user settable dose of a second drug agent and a variable set dose of a first drug agent to be delivered to the patient. The drug agents may be available in two or more reservoirs, containers or packages, each containing independent (single drug compound) or pre-mixed (co-formulated multiple drug compounds) drug agents. Activation of the needle guard automatically causes the reservoir of secondary medicament to engage with dispensing conduits to allow a set dose of primary medicament and a single fixed dose of the of the secondary medicament to be injected. Thus, a medicated module is presented where the user does not have to manually select or set the module to dispense the second drug agent. Secondary packaging for the medicated module is designed to prevent accidental triggering of the needle guard.

### BACKGROUND

Certain disease states require treatment using one or more different medicaments. Some drug compounds need to be delivered in a specific relationship with each other in order to deliver the optimum therapeutic dose. This invention is of particular benefit where combination therapy is desirable, but not possible in a single formulation for reasons such as, but not limited to, stability, compromised therapeutic performance and toxicology.

For example, in some cases it might be beneficial to treat a diabetic with a long acting insulin and with a glucagon-like peptide-1 (GLP-1), which is derived from the transcription product of the proglucagon gene. GLP-1 is found in the body and is secreted by the intestinal L cell as a gut hormone. GLP-1 possesses several physiological properties that make it (and its analogs) a subject of intensive investigation as a potential treatment of diabetes mellitus.

There are a number of potential problems when delivering two medicaments or active agents simultaneously. The two active agents may interact with each other during the long-term shelf life storage of the formulation. Therefore, it is advantageous to store the active components separately and only combine them at the point of delivery, e.g. injection, needle-less injection, pumps, or inhalation. However, the process for combining the two agents needs to be simple and convenient for the user to perform reliably, repeatedly and safely.

A further problem is that the quantities and/or proportions of each active agent making up the combination therapy may need to be varied for each user or at different stages of their therapy. For example, one or more actives may require a titration period to gradually introduce a patient up to a “maintenance” dose. A further example would be if one active requires a non-adjustable fixed dose while the other is varied in response to a patient's symptoms or physical condition. This problem means that pre-mixed formulations of multiple active agents may not be suitable as these pre-mixed formulations would have a fixed ratio of the active components, which could not be varied by the healthcare professional or user.

Additional problems arise where a multi-drug compound therapy is required, because many users cannot cope with having to use more that one drug delivery system or make the necessary accurate calculation of the required dose combination. This is especially true for users with dexterity or computational difficulties. In some circumstances it is also necessary to perform a priming procedure of the device and/or needle cannulae before dispensing the medicaments. Likewise, in some situations, it may be necessary to bypass one drug compound and to dispense only a single medicament from a separate reservoir.

Providing separate storage containers for two or more active drug agents that are only combined and/or delivered to the patient during a single delivery procedure allows for the delivery of two or more medicaments in a single injection or delivery step that is simple for the user to perform. This configuration also gives the opportunity for varying the quantity of one or both medicaments. For example, one fluid quantity can be varied by changing the properties of the injection device (e.g. dialing a user variable dose or changing the device's “fixed” dose). The second fluid quantity can be changed by manufacturing a variety of secondary drug containing packages with each variant containing a different volume and/or concentration of the second active agent. The user or healthcare professional would then select the most appropriate secondary package or series or combination of series of different packages for a particular treatment regime.

This configuration also provides a medicated module that automatically causes the reservoir of secondary medicament to come into fluid communication with the primary medicament upon activation of the needle guard. This eliminates the need for the user to manually set or adjust the medicated module after performing a priming step.

To prevent the medicated module from accidental activation, the module's secondary packaging comprises a mechanism to keep the module in a locked mode. Accidental triggering may occur prior to use, such as during transit or storage, and may either compromise the operability of the device, or render it unusable. Factors that may cause accidental triggering may include, but are not limited to, the application of static loads (e.g., stacking, crushing), dynamic loads (e.g., impact, vibration), pack and/or device inversion or temperature fluctuation.

Where accidental triggering has the potential to compromise the integrity of the Primary Pack, a patient may be exposed to a potentially non-sterile or even harmful form of the medicament.

Our invention seeks to prevent the accidental triggering of the medicated module. The act of removing the medicated module from its sterile packaging takes the module from a locked stated to a triggerable state. Thus, our invention is designed in such a way that the shift in the state from “trigger locked” to “triggerable” happens automatically as part of the standard, correct use procedure.

These and other advantages will become evident from the following more detailed description of the invention.

### SUMMARY

Our invention allows complex combinations of multiple drug compounds within a single drug delivery system. The invention allows the user to set and dispense a multi-drug compound device though one single dose setting mechanism and a single dispense interface. This single dose setter controls the mechanism of the device such that a predefined combination of the individual drug compound is delivered when a single dose of one of the medicaments is set and dispensed through the single dispense interface.

By defining the therapeutic relationship between the individual drug compounds, our delivery device would help ensure that a patient/user receives the optimum therapeutic combination dose from a multi-drug compound device without the inherent risks associated with multiple inputs where the user has to calculate and set the correct dose combination every time they use the device. The medicaments can be fluids, defined herein as liquids or powders that are capable of flowing and that change shape at a steady rate when acted upon by a force tending to change its shape. Alternatively, one of the medicaments may be a solid that is carried, solubilized or otherwise dispensed with another fluid medicament.

According to one specific aspect, this invention is of particular benefit to users with dexterity or computational difficulties as the single input and associated predefined therapeutic profile removes the need for them to calculate their prescribed dose every time they use the device and the single input allows considerably easier setting and dispensing of the combined compounds.

In a preferred embodiment, a master or primary drug compound, such as insulin, contained within a multiple dose, user selectable device could be used with a single use, user replaceable, module that contains a single dose of a secondary medicament and the single dispense interface. When connected to the primary device, the secondary compound is activated/delivered on dispense of the primary compound. Although our invention specifically mentions insulin, insulin analogs or insulin derivatives, and GLP-1 or GLP-1 analogs as two possible drug combinations, other drugs or drug combinations, such as an analgesics, hormones, beta agonists or corticosteroids, or a combination of any of the above-mentioned drugs could be used with our invention.

For the purposes of our invention the term “insulin” shall mean Insulin, insulin analogs, insulin derivatives or mixtures thereof, including human insulin or a human insulin analogs or derivatives. Examples of insulin analogs are, without limitation, Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

As used herein the term “GLP-1” shall mean GLP-1, GLP-1 analogs, or mixtures thereof, including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-Lys-Lys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

In one embodiment of our invention there is provided a medicated module attachable to a drug delivery device that comprises an outer housing having a proximal end, a distal end, and an outer surface, where the proximal end preferably has a hub holding a double-ended needle and having a connector configured for attachment to a drug delivery device. There is a reservoir in a bypass housing within the outer housing that contains a medicament. The medicated module assembly of our invention contains a needle guard that can reduce the risk of accidental needle sticks before and after use, reduce the anxiety of users suffering from needle phobia as well as preventing a user from using the device a subsequent time when the additional medicament has already been expelled.

The needle guard is preferably configured with a solid planar surface at its distal end that provides a large surface area that reduces the pressure exerted on the patient's skin, which allows the user to experience an apparent reduction in the force exerted against the skin. Preferably, the planar surface covers the entire distal end of the guard with the exception of a small needle pass through hole aligned axially with the needle. This pass through hole is preferably no more than 10 times greater in diameter than the outer diameter of the needle cannula. For example, with a needle outside diameter of 0.34 mm, the pass through hole diameter D can be 4 mm. Preferably, the pass through hole size should be large enough for the user to see that the device is primed (i.e., a drop or more of medicament) while not being so large that it is still possible to reach the end of the needle with a finger (i.e. needle stick injuries before or after use). This difference between the hole size and cannula diameter is to allow for tolerances, to allow users to see the drop of liquid on the end of the cannula after priming (whether a transparent or non-transparent guard is used) while keeping the size small enough to prevent accidental needle stick injuries.

Further, the movable needle guard or shield is configured to move axially in both the distal and proximal directions when pressed against and removed from an injection site. When the needle assembly is removed or withdrawn from the patient, the guard is returned to post-use extended position. A drive tooth on the inside surface of the guard engages a stop on a track on the outer surface of the bypass housing to securely lock the guard from further substantial axial movement. Preferably a lock out boss on the outer surface of the bypass housing is configured to engage a lock out feature on the inner proximal surface of the outer housing at the completion of the injection to further lock the medicated module from any further use and prevent the needle(s) and/or bypass component from being able to substantially move within the system even if the guard is held in an axially locked condition. By “substantial” movement we do not mean the typical amount of “play” in a system, but instead we mean that the guard and/or distal needle do not move axially a distance that exposes the distal end of the cannula once it is locked out.

One goal of our invention is to eliminate the need to have the user manually operate the medicated module to change the state of the module from a priming state to a combination dose delivery state. Manually operated devices are sometimes not as intuitive as they could be and raise the risk of accidental misuse. Our invention solves this problem by utilizing energy stored within the module prior to delivery of the device to the user. The stored energy can come from a biasing member, such as a compressed spring. This stored energy is released during normal user operation of the module by actuating the mechanism and thus activating the state change from prime dose to combination dose. The mechanism aims to make this actuation imperceptible to the user, consequently making the user experience of the module very similar to that of a standard commercially available and accepted needle or safety needle (i.e. unpack module, attach to a drug delivery device, prime drug delivery device, inject a set dose along with single dose in the module). In this way, the module mechanism aims to reduce the risk of unintentional misuse and to improve usability by replicating an already accepted practice for similar injection methods.

As the module mechanism does not require the user to access external features on the module for the purposes of actuation, the number of components and subsequent module size can be reduced/optimized. These factors make the mechanism ideal for a single-use, high-volume manufacture, and disposable device application. Alternatively, as the actuation is driven by a single energy source, the system lends itself to a resettable actuation mechanism. The preferred embodiment described below is the single use (non-resettable) version. The lower hub is preferably restrained rotationally with regard to the needle guard, but is free to move axially within the needle guard. The needle guard is restrained rotationally with regard to the outer housing, but is free to move axially, between defined constraints, within the outer housing.

The user pressing the distal face of the needle guard against the skin causes axial motion of the needle guard in the proximal direction. This axial motion of the guard causes a rotation of the bypass housing through the engagement and action of an inward-facing drive tooth on the guard as it travels in a drive track having one or more paths, which is located on the outer surface of the bypass housing. After sufficient axial travel of the needle guard, the rotation of the bypass housing brings stand-offs inside the outer housing and at the proximal ends of the lower hub into line with pockets located on the outer surface of the bypass housing. Alignment of the stand-offs with the pockets allows the bypass housing to move axially in the proximal direction and further into the outer housing. The lower hub containing a double-ended needle cannula moves axially further onto the bypass housing. Both of these movements occur due to the relaxation/release of the stored energy of the biasing member, preferably a spring that is pre-compressed during module assembly or manufacture, and constitute “triggering” of the actuation mechanism. It is this axial movement of the lower hub onto the bypass housing and the corresponding movement of the bypass housing further into the outer body that results in the double ended needles located in the outer body distal end and the lower hub piercing the medicated module, moving it from a state of priming to combination dose delivery.

Further axial movement of the needle guard is required in order to pierce the skin, this retraction of the needle guard temporarily re-compresses the biasing member creating additional stored energy. At a “commit” point, the proximal axial movement of the drive tooth passes a non-return feature in the track through further rotation of the bypass housing. In normal use, once the drug has been dispensed and the needle is removed from the skin, the needle guard is allowed to return axially in the distal direction under the relaxation of the biasing member as it releases its stored energy. At some point along its return travel, the drive tooth contacts a further ramped face in one of the paths of the track, resulting in yet further rotation of the bypass housing. At this point, the outer housing stand-off comes into contact with a ramp feature on the outer surface of the bypass housing. The combination of this feature with the ramp between the drive tooth and the bypass housing track results in further biasing of the bypass housing stop face into the needle guard drive tooth. The stop face features act as an axial locking pocket. The action of the combined biasing force means that any axial load in the proximal direction put on the needle guard will result in the tooth being stopped in this pocket, locking out the needle guard from further use or exposing the needle. Should the user remove the device from the skin without dispensing fluid, but after the “commit” point has been passed, the needle guard would return to an extended position and lock out as previously described.

In one embodiment of our invention there is provided a medicated module assembly attachable to a drug delivery device, preferably a pen shaped injection device, where the medicated module assembly comprises an outer housing having a proximal end and a distal end, where the proximal end has an upper hub holding a first double-ended needle cannula and a connector configured for attachment to a drug delivery device. The hub can be a separate part from the housing or integral, for example molded as part of the housing. The connector can comprise any connector design, such as threads, snap fits, a bayonet, a lure lock, or any combination thereof.

Two needle cannulae are used, a distal cannula and a proximal cannula, with both cannulae preferably being doubled-ended for piercing a septum or seal and for piercing skin. The distal needle is mounted in a lower hub and the proximal needle is mounted in the upper hub, each using a technique known to those skilled in the art, such as welding, gluing, friction fit, over-molding and the like. The medicated module assembly also contains a biasing member, preferably a torsion/compression spring. The biasing member is preferably in a pre-compressed state and positioned between the proximal inner face of the needle guard and the distal face of the lower hub. Although a preferred biasing member is a spring, any type of member that produces a biasing force will work.

The medicated module assembly of our invention automatically, once triggered, changes state from (1) a pre-use or priming state, where a small amount of primary medicament flows in a bypass around the reservoir containing a single dose of the secondary medicament, to (2) a ready-to-use or combination dose state, where both the upper and lower cannulae are in fluidic engagement with the fixed dose of the second medicament within the module and where a set dose of the primary medicament can be injected along with the non-settable single dose of secondary medicament in the reservoir, and finally to (3) a locked out state, where the needle guard is prevented from substantial proximal movement. The outer housing preferably has a window or indicator that shows the various states of the module. The indicator can be a pip, knob, button, or the like that protrudes through the outer surface of the proximal end of the needle guard and visually shows the user whether the module is in the pre-use or ready-to-use state. It may also be a visual indicator, e.g. showing colors or symbols, or a tactile or audible indicator. Preferably, user noticeable indicia indicate both a pre-use priming position and a locked position of the guard after the medicated module assembly has been used to perform an injection.

Inside the bypass housing there is a cavity that contains the capsule, which comprises the single dose of medicament in the reservoir. As the needle guard is retracted during an injection, the bypass housing is moved proximally along with the capsule positioned inside the cavity, thus decreasing the cavity volume. This allows the seals of the capsule to be pierced at its top and bottom by the needle cannula such that the medicament can be expelled from the reservoir during dose delivery. When connected to a drug delivery device containing a first medicament and prior to piercing the seals of the reservoir, the needle cannulae are only in fluid communication with the first medicament and a fluid flow path that bypasses the capsule. Preferably, a channel on the inside surface of the bypass housing is part of this fluid flow path and is used in the priming function of the drug delivery device.

As mentioned, the bypass housing preferably has one or more tracks located on the outside surface each having a set of first, second, third, and fourth paths. On the inner surface of the proximal end of the needle guard is one or more radial protrusions or drive teeth. As the guard first begins to retract, these protrusions travel in the first path, causing the bypass housing to slightly rotate. As the guard continues to retract and then partially extend, the protrusions travel in the second and third paths. The protrusion moves to the fourth path and into a locking position when the guard is fully extended to its post-use position, which is preferably less extended than the starting position. The guard is rotationally constrained by the outer housing, preferably by the use of one or more spline features in the outer surface of the guard in cooperation with one or more followers or pips located at the distal end of the inner surface of the outer housing. The bypass housing is rotationally constrained when the protrusion is in the second path of the track. As the protrusion is moved axially in the proximal direction when the guard retracts, the protrusion moves from the second track to the third track causing the assembly to emit an audile sound and/or tactile feedback. This tells the user that the device has now been activated to lock upon extension of the guard in the distal direction.

A further aspect of the invention relates to a method of dispensing a fixed dose of one medicament and a variable dose of a primary medicament from separate reservoirs that involves the steps of first attaching a medicated module to a delivery device set in a pre-use or prime only state. The user can prime the dose delivery device using only the primary medicament and bypassing the second medicament. After priming the user begins the injection and the needle guard begins to retract and the module automatically changes to second state that allows a combination delivery of the two medicaments. Upon completion of the delivery procedure and retraction of the needle from the injection site, the extension of the needle guard automatically changes the module to a third state.

During dispense, substantially the entire amount of second medicament has been expelled as well as the selected or dialed dose of the first medicament, through the single dispense interface. The capsule preferably contains a flow distributor to ensure that substantially all the single dose of secondary medicament is forced out of the capsule by the primary medicament during an injection. The flow distributor can be a separate stand alone insert or pin, or it may be integral with the capsule to make a one piece component utilizing, for example, design principles such as form fit, force fit or material fit, such as welding, gluing, or the like, or any combination thereof. The one-piece component may comprise one or more medicament flow channels, preferably one flow channel. The flow distributor can be constructed of any material that is compatible to the primary and secondary medicaments. A preferred material is one that is typically used to manufacture septa or pistons (bungs) found in multi-dose medicament cartridges, however, any other material that is compatible with the drug could be used, e.g., glass, plastics or specific polymers as described below. By “substantially all” we mean that at least about 80% of the second medicament is expelled from the drug delivery device, preferably at least about 90% is expelled. In the third state, preferably the module is locked so as to prevent a second delivery or insertion by means of a locking mechanism as described previously.

The combination of compounds as discrete units or as a mixed unit is delivered to the body via an integral needle. This would provide a combination drug injection system that, from a user's perspective, would be achieved in a manner that very closely matches the currently available injection devices that use standard needles.

The medicated module of our invention can be designed for use with any drug delivery device with an appropriate compatible interface. However, it may be preferable to design the module in such a way as to limit its use to one exclusive primary drug delivery device (or family of devices) through employment of dedicated/coded/exclusive features to prevent attachment of a non-appropriate medicated module to a non-matching device. In some situations it may be beneficial to ensure that the medicated module is exclusive to one drug delivery device while also permitting the attachment of a standard drug dispense interface to the device. This would allow the user to deliver a combined therapy when the module is attached, but would also allow delivery of the primary compound independently through a standard drug dispense interface in situations, such as, but not limited to, dose splitting or top-up of the primary compound.

A particular benefit of our invention is that the medicated module makes it possible to tailor dose regimes when required, especially where a titration period is necessary for a particular drug. The medicated module could be supplied in a number of titration levels with obvious differentiation features such as, but not limited to, aesthetic design of features or graphics, numbering etc, so that a patient could be instructed to use the supplied medicated module in a specific order to facilitate titration. Alternatively, the prescribing physician may provide the patient with a number of “level one” titration medicated modules and then when these were finished, the physician could then prescribe the next level. A key advantage of this titration program is that the primary device remains constant throughout.

In a preferred embodiment of our invention, the primary drug delivery device is used more than once and therefore is multi-use; however, the drug delivery device may also be a single use disposable device. Such a device may or may not have a replaceable reservoir of the primary drug compound, but our invention is equally applicable to both scenarios. It is also possible to have a suite of different medicated modules for various conditions that could be prescribed as one-off extra medication to patients already using a standard drug delivery device. Should the patient attempt to reuse a previously used medicated module, our invention includes the locking needle guard that is activated after a first predefined travel/retraction of the guard/insertion of the needle. The locked needle guard would alert the patient to this situation and the inability to use the module for a second time. Visual warnings (e.g. change in color and/or warning text/indicia within an indication window on the module once insertion and/or fluid flow has occurred) can also be used. Additionally, tactile feedback (presence or absence of tactile features on the outer surface of the module hub following use) could be used as well.

A further feature of our invention is that both medicaments are delivered via one injection needle and in one injection step. This offers a convenient benefit to the user in terms of reduced user steps compared to administering two separate injections. This convenience benefit may also result in improved compliance with the prescribed therapy, particularly for users who find injections unpleasant or who have computational or dexterity difficulties.

Our invention also covers a method of delivering two medicaments stored in separate primary packages. The medicaments may both be liquid, or alternatively one or more of the medicaments may be a powder, suspension or slurry. In one embodiment the medicated module could be filled with a powdered medicament that is either dissolved or entrained in the primary medicament as it is injected through the medicated module.

Furthermore, our invention is also directed to secondary packages and packaging accessories for storing and transporting the modules, such as medicated modules. The secondary packages are designed with features in the packaging that interact with the module while the module is within the packaging, preventing movement of the body of the device relative to the needle guard, such that the module is in a “trigger locked state.” When the module is removed from the secondary packaging, the features become removed from the module and thus transitioning the module to a “triggerable” state where it is ready to be used. In one example a module may at least be partly covered by a secondary packaging. The module may have an initial state or “trigger locked state” and an actuated state or “triggerable state”. The module may comprise a guard and at least one restraining element having a first position, preventing movement of the guard and a second position allowing movement of the guard. The cover may be arranged to conform with the exterior of the module so as to restrain elements of the module from moving to each other. In a further example movement of at least a portion of the cover relative to the module brings the restraining element from the first position to the second position thereby changing state of the module from the initial state to the actuated state. In another example the secondary packaging, such as a cover, is in the form of a container comprising a cavity portion and a closure member attached thereto and which together form an enclosure in which the module is arranged initially. Movement of the closure member relative to the module brings the restraining element from the first position to the second position thereby changing state of the module from the initial state to the actuated state.

One purpose of such an arrangement is to ensure that stored energy present in the module, such as an energized biasing member described above, is protected from external interference/influence and minimizes the likelihood of accidental triggering of the device and release of this stored energy up until the point that the user removes it from its packaging for conscious use.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates one possible drug delivery device that can be used with the present invention;

FIG. 2 illustrates an embodiment of the medicated module of the present invention, where the medicated module is separated from an attachable cartridge holder of drug delivery device;

FIG. 3 illustrates an exploded distal perspective view of all the components (except the medicated capsule) of the medicated module illustrated in FIG. 2;

FIG. 4 illustrates an exploded proximal perspective view of all the components (except the medicated capsule) of the medicated module illustrated in FIG. 2;

FIG. 5 is a perspective view of the capsule containing the reservoir of the embodiment of FIG. 2;

FIG. 6 illustrates a proximal perspective view of the outer housing of the embodiment of FIG. 2;

FIG. 7 is a sectioned view of the embodiment of the medicated module shown in FIG. 2 orientated in the bypass configuration;

FIG. 8 is a close-up perspective view of the bypass housing of the embodiment of the medicated module shown in FIG. 2 to illustrate the positions of the drive tooth during use;

FIG. 9 is a perspective view of an exemplary module within a secondary packaging in a trigger locked position;

FIG. 10 is a side view of the module within the secondary packaging of FIG. 9, in the triggerable position;

FIG. 11 is a perspective view of an exemplary module, wherein the module is in a trigger locked position;

FIG. 12 is a perspective view of an exemplary module in the triggerable position;

FIG. 13 is a side view of an exemplary module within a secondary packaging in a trigger locked position;

FIG. 14 is a side view of the module and secondary packaging in a triggerable position;

FIG. 15 is a perspective view of an exemplary module;

FIG. 16 is a cross-sectional view of an exemplary module;

FIG. 17 is a top view of the medicated of FIG. 15 within a secondary packaging;

FIG. 18 is a side view of the packaging and module in the trigger locked position;

FIG. 19 is a side view of an exemplary module within a secondary packaging in a trigger locked position;

FIG. 20 is a side view of the module within the secondary packaging of FIG. 9, in the triggerable position;

FIG. 21 is a side view of an exemplary module within a secondary packaging in a trigger locked position;

FIG. 22 is a side view of the module within the secondary packaging of FIG. 9, in the triggerable position;

FIGS. 23-25 illustrate cross-sectional views of an exemplary module;

FIGS. 26-28 illustrate alternate views of yet another exemplary embodiment of a module with restraints;

FIGS. 29-31 illustrate yet another exemplary embodiment of a module and a secondary packaging;

FIGS. 32-34 illustrate yet another embodiment of a module for use with secondary packaging;

FIG. 35 illustrates a perspective view of yet another embodiment of a module;

FIG. 36 illustrates a cross-sectional view of yet another embodiment of a module for use with secondary packaging;

FIG. 37 illustrates a perspective view of the module illustrated in FIG. 36;

FIG. 38 illustrates a partial, cross-sectional view of a secondary packaging;

FIG. 39 illustrates a perspective view of an alternative module placed within the secondary packaging illustrated in FIG. 38;

FIG. 40 illustrates a partial, cross-sectional view of an alternative secondary packaging;

FIG. 41 illustrates a perspective view of an alternative module placed within the secondary packaging illustrated in FIG. 40;

FIG. 42 illustrates a perspective view of an exemplary embodiment of a module within a secondary packaging;

FIG. 43 illustrates a perspective view of the module illustrated in FIG. 42 being removed from the secondary packaging;

FIG. 44 illustrates a cross-sectional view of the module within the secondary packaging in the position illustrated in FIG. 42; and

FIG. 45 illustrates a cross-sectional view of the module being removed from the secondary packaging illustrated in FIG. 42.

### DETAILED DESCRIPTION

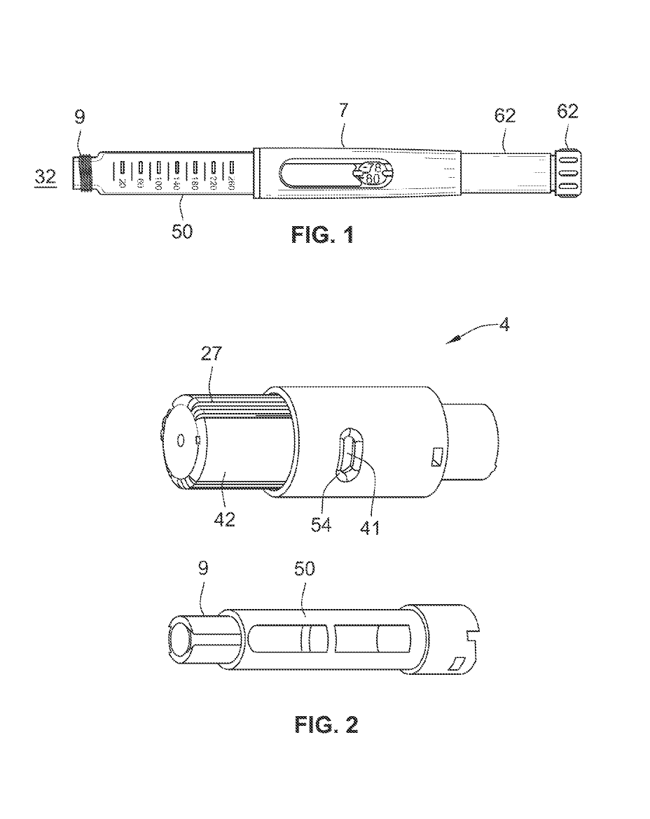

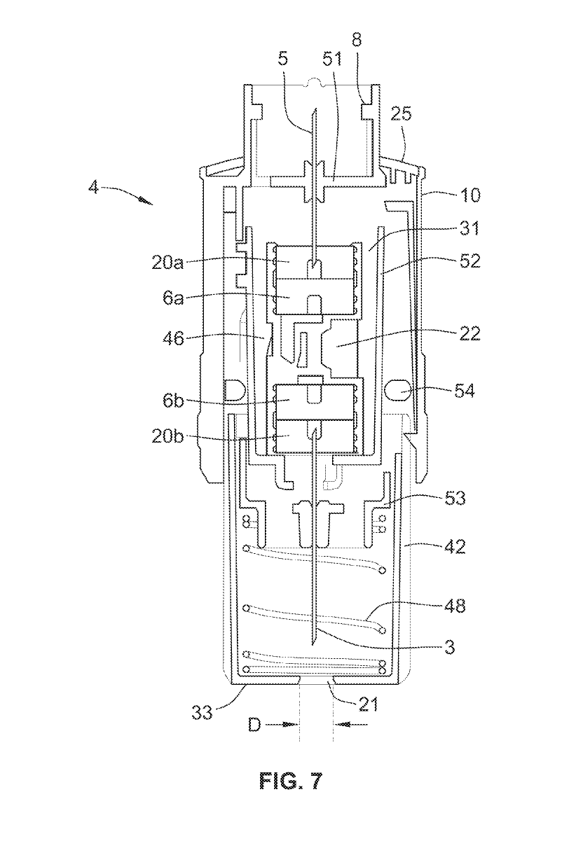

The present invention provides a locking mechanism for a medicated module and secondary packaging for the medicated module. The medicated module administers a fixed predetermined dose of a secondary drug compound (medicament) and a variable dose of a primary or first drug compound through a single output or drug dispense interface. Setting the dose of the primary medicament by the user automatically determines the fixed dose of the second medicament, which preferably is a single dose contained in a capsule or reservoir having an integral flow distributor. In a preferred embodiment the drug dispense interface is a needle cannula (hollow needle). FIG. 1 illustrates one example of a drug delivery device 7 that the medicated module 4 (see FIG. 2 or 7) can be attached to. The medicated module can be attached by the connection means 9 on distal end 32 of cartridge holder 50. Each medicated module is preferably self-contained and provided as a sealed and sterile disposable module that has an attachment means 8 compatible to the attachment means 9 at the distal end 32 of device 7.

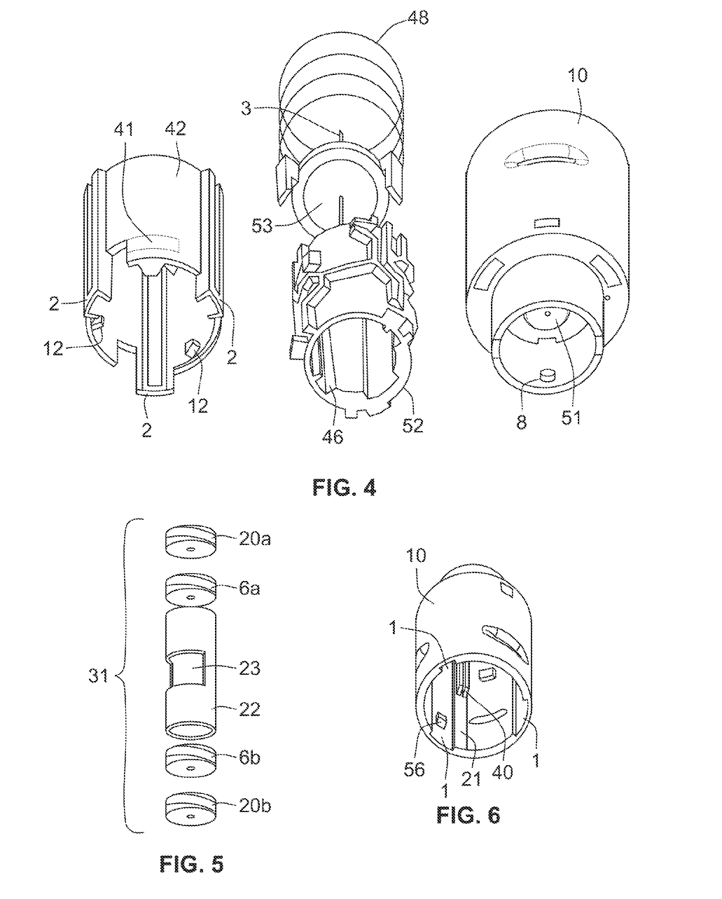

Any known attachment means 8 can be used to attach the medicated module to the chosen drug delivery device, including all types of permanent and removable connection means, such as threads, snap locks, snap fits, luer locks, bayonet, snap rings, keyed slots, and combinations of such connections. FIGS. 2, 4, and 7 illustrate the attachment means 9 as a unique bayonet type connection that is keyed specifically to a corresponding female bayonet type connection 8 on hub 51 of medicated module 4. The embodiments shown in FIGS. 2, 4, 5, and 7 have the benefit of the second medicament as a single dose being contained entirely within capsule 31, and specifically in reservoir 22, hence minimizing the risk of material incompatibility between the second medicament and the materials used in the construction of the medicated module 4, specifically housing 10, inner housing 52, or any of the other parts used in the construction of the medicated module.

To minimize the residual volume of the second medicament, caused by recirculation and/or stagnant zones, that might remain in capsule 31 at the end of the dispense operation, it is preferable to have a flow distributor 23 as an integral part of reservoir 22 (see FIG. 5). The reservoir 22 containing the single dose of the secondary medicament can be sealed with septa 6a and 6b, which are fixed to the capsule using keepers or plugs 20a and 20b. Preferably the keepers have fluid channels that are in fluid communication with needles 3 and 5 and with bypass 46, which is preferably part of the inside surface of bypass housing 52. Together this fluid path allows priming of the drug delivery device before injection. Preferably the reservoir, flow distributor, keepers, and bypass can be made from materials that are compatible with the primary medicament. Examples of compatible materials of construction include, but are not limited to, COC (an amorphous polymer based on ethylene and norbonene, also referred to as cyclic olefin copolymer, ethylene copolymer, cyclic olefin polymer, or ethylene-norbornene copolymer); LCP (a liquid crystal polymer having an aramid chemical structure that includes linearly substituted aromatic rings linked by amide groups, and further can include partially crystalline aromatic polyesters based on p-hydroxybenzoic acid and related monomers and also highly aromatic polyesters); PBT (polybutylene terephthalate thermoplastic crystalline polymer or polyester); COP (a cyclic olefin polymer based on ring-opening polymerization of norbornene or norbornene-derivatives); HDPE (high density polyethylene); and SMMA (styrene methyl methacrylate copolymer based on methyl methacrylate and styrene). The needle pierceable septa, bungs, and/or seals that are used with both the capsule and the primary medicament cartridge can be manufactured using TPE (thermo plastic elastomer); LSR (liquid silicone rubber); LDPE (low density polyethylene); and/or any kind of medical grade rubber, natural or synthetic.

The design of flow distributor 23 should ensure that at least about 80% of the second medicament is expelled from reservoir 22 through the distal end of needle 3. Most preferably at least about 90% should be expelled. Ideally, displacement of the first medicament in a primary reservoir (not shown) contained in cartridge holder 50 and through the capsule 31 will displace the single dose of the second medicament stored in reservoir 22 without substantial mixing of the two medicaments.

Attachment of the medicated module 4 to the multi-use device 7 causes proximal needle 5 to penetrate a septum (not shown) sealing the distal end of the cartridge of primary medicament positioned in cartridge holder 50 of the multi-use device 7. Once needle 5 has passed through the septum of the cartridge, fluid connection is made between the first medicament and the needle 5. At this point, the system can be primed by dialing out a small number of units (or cocking the device if only a single dose selection is possible) using dose dial sleeve 62. One the device 7 is primed, then activation of the needle guard 42 allows dispense of the medicaments by subcutaneously injecting the medicaments via activation of a dose button 13 on device 7. The dose button of our invention can be any triggering mechanism that causes the dose of the first medicament that was set by the dose dial sleeve 62 to move towards the distal end 32 of the device. In a preferred embodiment the dose button is operably connected to a spindle that engages a piston in the primary reservoir of the first medicament. In a further embodiment the spindle is a rotatable piston rod comprising two distinct threads.

One embodiment of the medicated module 4 is illustrated in FIGS. 2 and 7. In these embodiments the medicated module 4 contains a capsule 31 comprising a reservoir 22, two keepers 20a and 20b, and two seals 6a and 6b. Reservoir 22 contains a fixed single dose of a secondary medicament. In some cases this secondary medicament may be a mixture of two or more drug agents that can be the same or different from the primary drug compound in the drug delivery device 7. Preferably the capsule is permanently fixed within the medicated module, however, in some cases it may be preferred to design the module such that the capsule can be removed when empty and replaced with a new capsule.

In the embodiments shown in FIGS. 5 and 7, capsule 31 has ends that are sealed with pierceable membranes or septa 6a and 6b that provide a hermetically sealed and sterile reservoir 22 for the second medicament. A primary or proximal engagement needle 5 can be fixed in hub 51 connected to the proximal end of housing 10 of the module and configured to engage capsule 31 when needle guard is moving in the proximal direction during injection. The outlet, or distal needle 3, is preferably mounted in lower hub 53 and initially protrudes into lower keeper 20b. The proximal end of needle 3 pierces the lower septum 6b when the bypass housing 52 rotates and is moved proximally by the force exerted by needle guard 42 and spring 48 during injection.

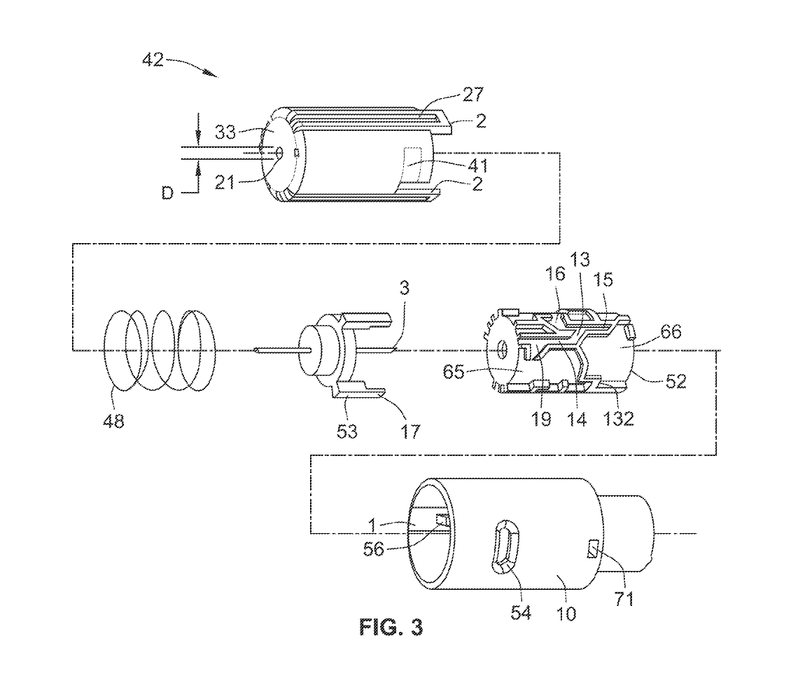

When first attached to the delivery device, the medicated module 4 is set at a pre-use or starting position. Preferably, indicator 41 shows through window 54 to inform the user of the pre-use condition of the medicated module. The indicator is preferably a color stripe or band on the outer surface of the proximal end of guard 42 (see FIG. 3) visible through an aperture in the outer body. The needle guard 42 is slidably engaged with inner surface of outer housing 10 by engagement of arms 2 and channels 1. Retention snaps 56 prevent the guard from disengaging the outer housing at its fully extended position. Housing 10 partially defines an internal cavity 21 that holds bypass housing 52, which contains capsule 31. A portion of the proximal end of housing 10 defines an upper hub 51 that holds needle 5. Optionally, as illustrated in FIG. 7, a shoulder cap 25 may be added to the proximal outer surface of outer housing 10. This shoulder cap can be configured to serve as indicia to identify to a user the type/strength of medicament contained in the module. The indicia can be tactile, textual, color, taste or smell.

FIG. 7 shows a cutaway or cross-sectioned view of the medicated module set in a pre-use or starting state where needles 3 and 5 are not piercing septa 6a and 6b. In this position, the bypass housing 52 is at its most extended position and needles 3 and 5 are not in fluid communication with medicament contained in capsule 31. The capsule is supported by bypass housing 52. In this neutral or suspended state of capsule 31, primary medicament from the cartridge in cartridge holder 50 of device 7 can flow through needle 5 into keeper 20a, through bypass 46 and into keeper 20b, and eventually out through needle 3. This flow configuration allows a user to perform a priming step or procedure by setting a small dose of the primary medicament using the dose dial sleeve 62 and dose button 13 on the drug delivery device 7.

The compression spring 48 is positioned between the distal end of bypass housing 52 and the inner proximal face of guard 42 to bias the guard 42 into an extended (guarded) position as illustrated in FIG. 7. Upon assembly, spring 48 is purposely compressed to supply a proximally directed biasing force against lower hub 53. This pre-compression of spring 48 is possible because the lower hub 53 and the bypass housing 52 are prevented from moving in an axial proximal direction by radial stand off 40 located on the inside surface of the outer housing (FIG. 6) that engage with an upper stand off pocket 66 and legs 17 of lower hub 53 engaging lower stand off pocket 65. The combination of these stand-offs/legs and pockets prevent the lower hub and upper hub needles from piercing into the centre of the capsule until the device is triggered as previously described.

The proximal inside surface of guard 42 has one or more inwardly protruding features, drive teeth, pips, or like structures 12 that run in one or more tracks 13 or guide ways formed in the outer surface of bypass housing 52. As shown in FIG. 3, track 13 can be described as four paths, 19, 14, 15, and 16, that have a specific geometry such that after a single use of the medicated module 4 the drive tooth 12 is blocked from further axial movement and the guard (and device) is “locked” in a guarded position where the distal end of the needle is completely and safely covered by guard 42.

One unique feature of our medicated module assembly is the user feedback that is given when the assembly is used. In particular, the assembly could emit an audible and/or tactile “click” to indicate to the user that they have firstly triggered the device and secondly reached the “commit” point such that the needle guard will lock safely out upon completion of the injection/removal of the guard from the injection site. This audible and/or tactile feature could work as follows. As mentioned, the needle guard 42 is rotationally constrained by outer housing 10 and has one or more drive teeth 12 that are initially in path 19 of track 13 on bypass housing 52. As the guard is moved proximally, the spring 48 is further compressed exerting additional force in the proximal direction on lower hub 53, which is initially constrained axially by the lower stand off pocket 65 engaged with legs 17. Likewise, the bypass housing 52 is constrained from moving proximally by upper stand off pocket stop 132 engaged with stand off 40 on the inner surface of outer hosing 10. The drive teeth 12 travel in path 19 causing the bypass housing to rotate slightly. This rotation will disengage the upper stand off 40 from upper standoff pocket stop 132, allows the drive teeth to enter path 14, and unblocks legs 17 from lower standoff pocket allowing the bypass housing to move proximally carrying with it capsule 31, where it then can engage needles 3 and 5. As the guard continues to move proximally, the drive teeth move from path 14 passed transition point 14a into path 15 causing further rotation of the bypass housing. As this rotation is completed the drive teeth transition to path 13, potentially emitting an audile “click” sound, as well as a tactile feel, to the user. This transition past point 15a (and the corresponding point directly below it on the track) constitute the “commit” point and as such, once it has been reached the needle guard 42 will “lock out” when it extends upon removal of the device from the injection site.

As mentioned, the distal end of the guard 42 has a planar surface 33 that provides an added measure of safety and reduces the pressure exerted by the guard on the injection site during an injection with our needle assembly. Because the planar surface 33 substantially covers access to needle 3 a user is prevented from gaining access to the distal tip of the needle after the assembly is in the locked position. Preferably, the diameter D of needle pass through hole 21 in the planar surface is no more than 10 times that of the outer diameter of needle cannula 3.

The outer proximal surface of the needle guard 42 preferably has indicia 41 that are preferably at least two different color stripes or bands, each of which is sequentially visible through the opening or window 54 in outer housing 10. One color could designate the pre-use or prime state of the module and the other color would indicate that the module is in finished or locked state, another color could be used to denote the transition through the trigger or “commit” point in case a user stops injection after trigger point but before “commit” point. For example, a green color could be the pre-use position and a band of red color could be used to indicate that the module has been used and is locked and an orange color could indicate that the device has been triggered but not locked out. Alternatively, graphics, symbols or text could be used in place of color to provide this visual information/feedback. Alternatively these colors could be displayed using the rotation of the bypass cavity and printed on or embedded into the bypass housing. They could be visible through the aperture by ensuring that he needle guard is made form a transparent material.

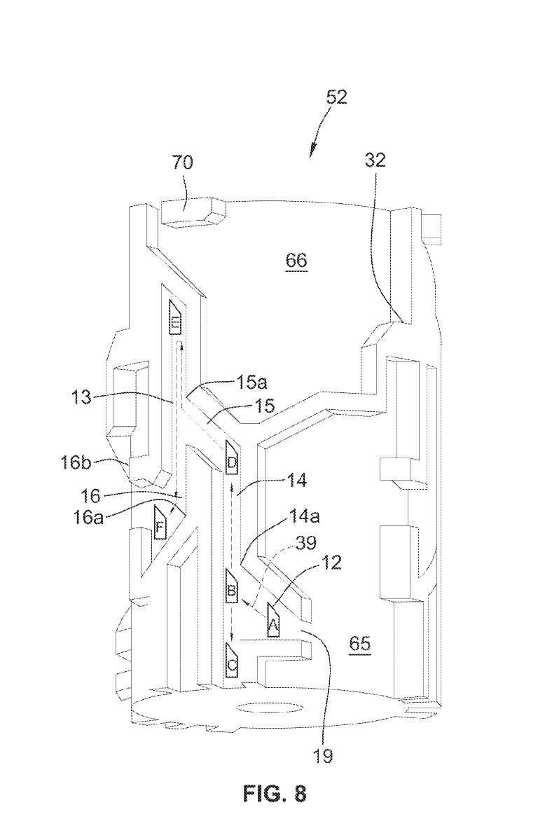

FIG. 8 illustrates the travel of drive teeth 12 in one or more tracks 13 as illustrated by directional arrow 39. Drive tooth 12 begins at position A and through axial movement of the needle guard, biases the bypass housing rotationally until it moves past the transition point 14a and arrives at position B. Once the drive tooth reaches position B, the bypass housing and lower needle hub move proximally causing the capsule 31 to engage needles 3 and 5, and the drive tooth moves relatively to position C (this is termed as the triggering of the device) and it is the bypass housing/lower hub moving proximally under the release of stored energy that results in the effective position of the needle guard drive tooth being position C. It is important to note that the needle guard does not move under the action of the release stored energy, it is just the needle hub and the bypass housing that move relatively away from the needle guard at the point of triggering, hence the drive tooth moves from position B to position C. As the needle guard continues to retract, drive tooth 12 moves proximally in path 14 to position D, where it exerts a rotational bias on the bypass housing 52, causing it to rotate again until tooth 12 passes the transition 15a (commit point) into path 16. The drive tooth then moves proximally until position E is reached. At this point, the needle guard 42 is fully retracted and the full available insertable length of the needle is exposed. Once the user removes the guard from contact with the skin, the guard begins to extend as a result of the distal biasing force exerted by spring 48 on the inner proximal surface of the guard. The utilization of the stored energy spring to act both as a trigger/piercing spring and also, once extended post triggering, as the needle guard spring, is a unique aspect of this design. It negates the need to use two separate springs for these separate functions by locating the spring in a position such that it can fulfill both roles. Initially, for example during assembly or manufacture of the medicated module, the biasing member is compressed, exerting a force on the lower hub/bypass housing in preparation for triggering. Once triggered it extends proximally where upon it can then be compressed from the distal end as the needle guard retracts against it. This secondary compression provides the force to push the needle guard back to the extended and locked position as it is removed from the injection site. As the guard moves to its fully extended post-use position, which preferably is less extended than the starting position, the drive tooth 12 moves distally in path 15 until it reaches transition point 16a, where it then rotationally biases the bypass housing 52 to rotate yet again until tooth 12 enters path 16 and arrives at position F. This last rotation of bypass housing 52 causes lock out boss 70 to engage lock out feature 71. This prevents any further rotational or axial movement of the bypass housing. The needle guard is prevented from further substantial axial movement, as defined earlier, by engagement of the drive tooth with axial stop 16b. It is within the scope of our invention that a number of tooth arrangements and/or profiles could be used to fulfill the required function described above, e.g., simple equal tooth profiles or more complex multi-angled profiles. The particular profile being dependent upon the required point of commit and rotation of the bypass housing. It is also within the scope of our invention that a similar axial/rotational locking of the lower needle hub to the bypass housing as of the bypass housing to the outer housing, could be integrated to prevent movement of the needle post-triggering and post-lock out.

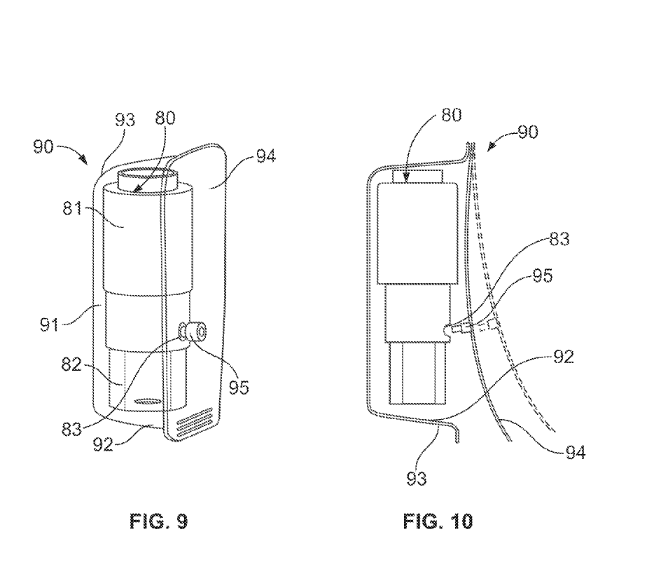

FIG. 9 is a perspective view of an exemplary module 80, such as the medicated module 4 illustrated in FIGS. 2 and 7, within a secondary packaging 90 in a trigger locked position.

In this embodiment, module 80 may comprise at least some of the same components as those described for the medicated module 4 of FIGS. 2 and 7. Module 80 comprises a device 81, a needle guard 82, and an indent or hole 83. Hole 83 preferably extends through both device 81 and needle guard 82.

Secondary packaging 90 comprises a main body 91 with an interior surface 92, an exterior surface 93, and a lid 94. In one embodiment, a peg 95 extends from interior surface 92 of lid 94 but the peg could be mounted anywhere within the packaging that allows easy removal of the device from the package.

After module 80 is placed within secondary packaging 90, lid 94 is closed and peg 95 of packaging 90 enters into hole 83 of the module. FIG. 9 shows peg 95 fully inserted into hole 83. While peg 95 is within hole 83, peg 95 extends through both device 81 and needle guard 82, blocking and preventing axial movement of the needle guard 82 relative to the device 81. This allows peg 95 to maintain the medicated module 80 in a locked, non-triggerable position.

FIG. 10 is a side view of the module within the secondary packaging of FIG. 9. In this view, lid 94 is opened away from module 80. When lid 94 is pulled open, peg 95 exits hole 83, removing the block that prevented needle guard 82 from moving axially with respect to device 81. Thus, needle guard 82 can now move, and module 80 is in the triggerable position.

The system illustrated in FIGS. 9-10 may be re-usable such that whenever peg 95 is inserted into module 80, the module once again is in the trigger locked position.

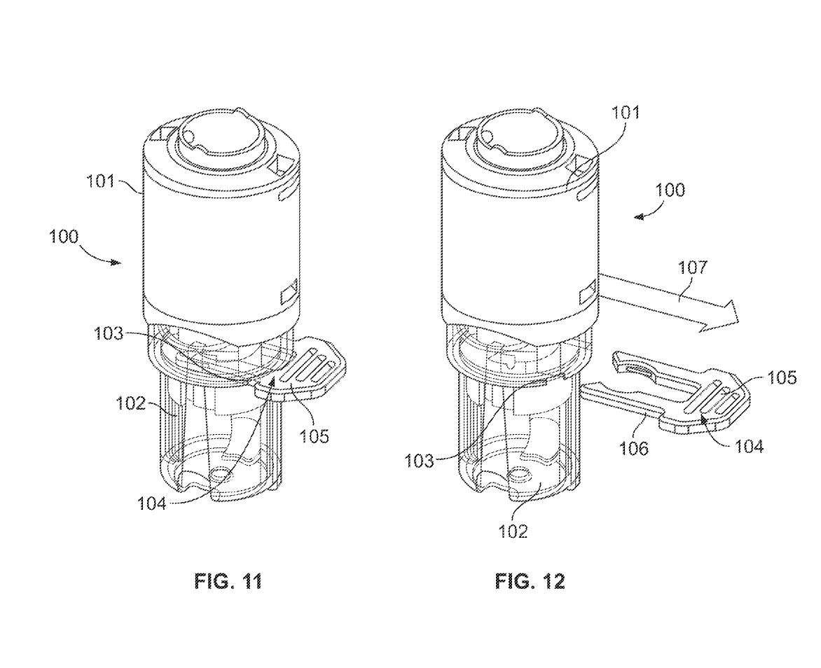

FIG. 11 is a perspective view of an exemplary module 100 in a trigger locked position, such as the medicated module 4 illustrated in FIGS. 2 and 7.

In this embodiment, module 100 may comprise at least some of the same components as those described for the medicated module 4 of FIGS. 2 and 7. Module 100 comprises a device 101, a needle guard 102, and a pair of slits 103. Slits 103 preferably extend through both device 101 and needle guard 102.

A pin 104, such as a grenade pin, for example, is inserted into slits 103 as shown in FIG. 11. Pin 104 preferably comprises a gripping portion 105, and a pair of extensions 106 (shown in FIG. 12). When pin 104 is fully inserted into slits 103, such that extensions 106 are inserted into the module 100, the extensions block needle guard 102 from axial movement relative to the device 101. This is the trigger locked position.

As pin 104 is pulled in the direction shown by arrow 107, extensions 106 are removed from slits 103. Once extensions 106 are completely removed from the module 100, needle guard 102 can move in relation to the device 101. Thus, FIG. 12 shows the module in the triggerable position.

The system illustrated in FIGS. 11-12 may be re-usable such that whenever pin 105 is inserted into module 100, the module once again is in the trigger locked position.

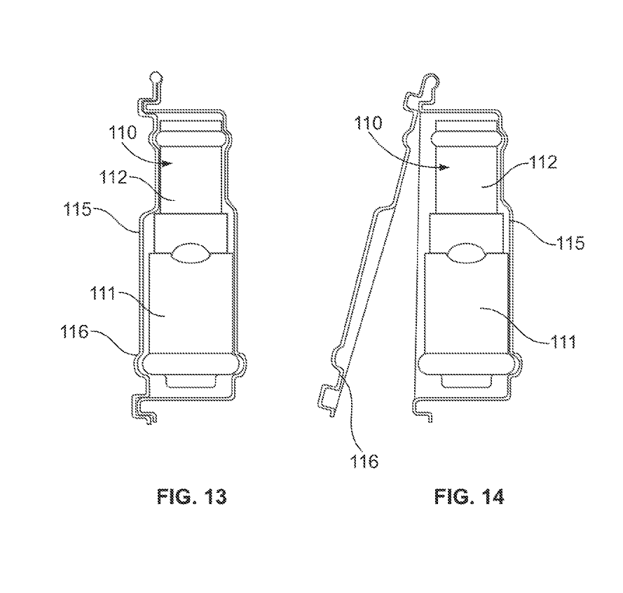

FIG. 13 is a side view of an exemplary module 110 within a secondary packaging 115. As can be seen in FIG. 13, the secondary packaging 115 is contoured to substantially conform to the external module 110 features, preventing the needle guard 112 and the device 111 from moving relative to one another. This is the trigger locked position.

Secondary packaging 115 comprises a lid 116, that, when opened (as shown in FIG. 14), allows for the module 110 to be removed. Once module 110 is removed from secondary packaging 115, it can be triggered and the needle guard 112 and device 111 can move relative to one another.

The system illustrated in FIGS. 13-14 may be re-usable such that whenever module 110 is within the closed secondary packaging 115, the module once again is in the trigger locked position.

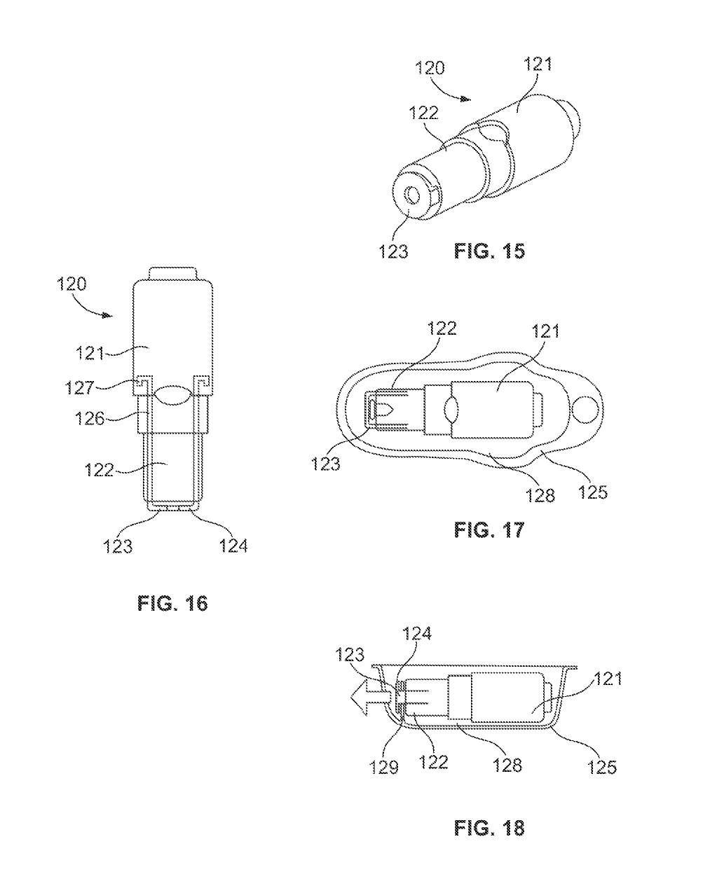

FIG. 15 is a perspective view of an exemplary module 120, such as the medicated module 4 illustrated in FIGS. 2 and 7. Module 120 comprises a device 121, a needle guard 122, and a secondary guard 123. Secondary guard 123 comprises a first end portion 124, and a pair of extensions 126 that prevent the axial travel of the needle guard relative to the device body until such a time as the secondary guard is pressed axially against the distal outer face of the needle guard, whereby the device is in a ‘triggerable’ condition, each extension having a hook-shaped second end portion 127 as shown in the cross-sectional view of FIG. 16. The extensions preferably extend substantially orthogonally from first end portion 124. First end portion 124 may be shaped to be circular member with a hole in the center of the member, to allow for a needle to pass through.

In order to activate the module such that it is in a triggerable position, the first end portion 124 is pressed against a user's skin. Secondary guard 123 is held outwards by a feature in the secondary packaging, thus ensuring that it cannot move axially until the device is removed from the packaging. Pressing the module against the injection site, such as the skin, actuates the extensions, thereby releasing the needle guard.

In an alternative configuration, FIG. 17 illustrates the medicated module 120 of FIG. 15, wherein the module 120 interfaces with a secondary packaging 125. Secondary packaging 125 comprises a main body with an interior surface 128. A flange 129 extends essentially orthogonally from the interior surface 128 When module 120 is placed within secondary packaging 125, flange 129 is positioned between first end portion 124 and the end of the needle guard 122. Thus, flange 129 keeps first end portion 124 of secondary guard 123 from moving back toward the needle guard 122, thus keeping medicated module in a trigger locked position. In this arrangement, first end portion 124 is biased to spring inwards and the flange 129 prevents the end portion from spring inwards. Therefore, when the device is removed from the packaging and end portion 124 disengages from flange 129, the end portion 124 pops inwards automatically and makes the device triggerable. In this manner, the device is kept safe in the secondary packaging and remaining automatically “armed” through the action of removing it from this packaging.

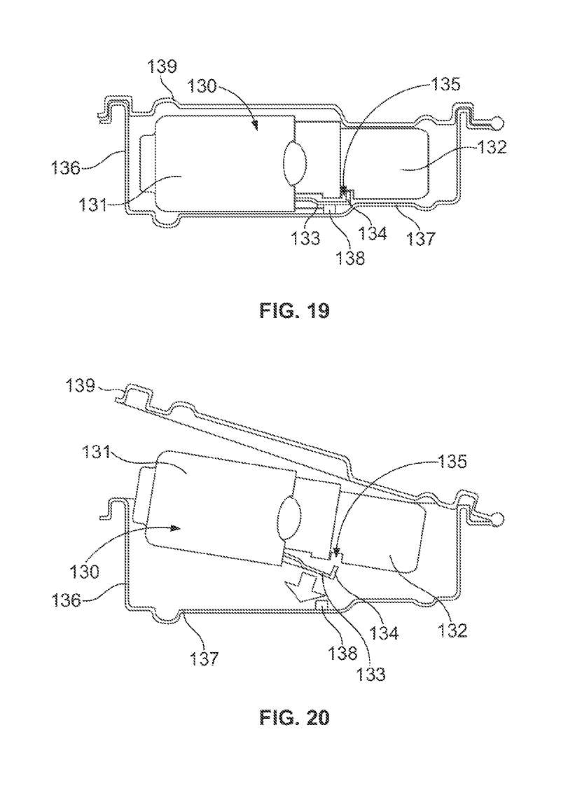

FIG. 19 is a side view of an exemplary module 130 within a secondary packaging 136, such as the medicated module 4 illustrated in FIGS. 2 and 7. Module 130 comprises a device 131, a needle guard 132, and a sprung latch 133. Sprung latch may be attached to device 131, and may comprise a hook member 134 that fits within an indent 135 in needle guard 132.

In FIG. 19, module 130 is in the trigger locked position within secondary packaging 136. Secondary packaging 136 comprises an interior surface 137, an extension 138 extending from interior surface 137, and a lid 139. In this position, extension 138 from the interior of secondary packaging presses against sprung latch 133, compressing sprung latch 133. In the compressed state, hook member 134 of sprung latch 133 fits within indent 135 of needle guard 132. When sprung latch 133 is in indent 135, needle guard 132 cannot move with respect to device 130. Sprung latch 133 keeps needle guard 133 in the trigger locked position.

FIG. 20 is a side view of the module 130 of FIG. 19, in the triggerable position. When lid 139 is opened and module 130 is removed from packaging 136, sprung latch 133 no longer is compressed and retained in position within indent 135 by extension 138. Sprung latch 133 moves into its relaxed position, with latch 133 moving out of indent 135. Module 130 is now in the triggerable position, and needle guard 132 can move with respect to device 131.

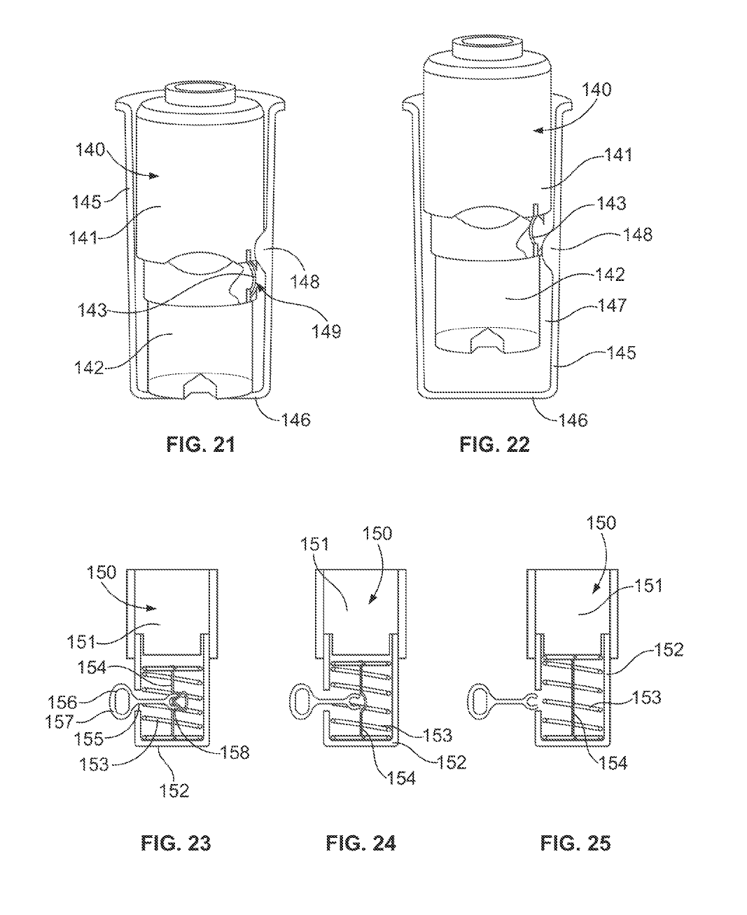

FIG. 21 is a cross-sectional view of an exemplary module 140 within a secondary packaging 145, such as the medicated module 4 illustrated in FIGS. 2 and 7. Module 140 comprises a device 141, a needle guard 142, and a bi-stable spring 143. Secondary packaging 145 comprises a cylindrical main body 146, with an interior surface 147, and a bump or extension 148 along interior surface 147. Bump 148 is convex to the interior surface 147 of packaging 145. When popped out, the spring 143 passes through an aperture 149 in the body 146 as may be seen in FIG. 21. If the needle guard 142 experiences an axial load, the spring 143 will clash with the edges of the aperture (appear as small triangles in the FIG. 21) which bite into the spring element 143. When the spring 143 is reversed, it is well clear of the body aperture 149 and does not interfere when the needle guard 142 is moved axially.

When module 140 is in the trigger locked position shown in FIG. 21, bi-stable spring 143 is pushed outward, away from module 140. In this position, spring 143 prevents the relative motion of the needle guard 142 that would trigger device 141.

In FIG. 22, which is a cross-sectional view of the module 140 and secondary packaging 145, module 140 is pulled partially out of packaging 145. As module 140 is pulled out of packaging 145, bi-stable spring 143 moves over bump 148. The pressure bump 148 exerts on spring 143 deflects spring 143, causing spring 143 to invert, and spring 143 now is stable in the inverted position shown in FIG. 22. Module 140 is now in the triggerable position, as spring 143 no longer stands in the way of needle guard 142 moving in relation to device 141.

FIGS. 23-25 are cross-sectional views of an exemplary module 150, such as the medicated module 4 illustrated in FIGS. 2 and 7. Module 150 comprises a device 151, a needle guard 152, a compression spring 153, and a restraint 154. Needle guard 152 comprises an opening 155. A pin 156 with a handle 157 and a clenching member 158 resides in opening 155, as shown in FIG. 23, when the medicated module 150 is in the trigger locked position. Restraint 154 may be a wire that extends in the axial direction across at least a portion of spring 153.

In FIG. 23, handle 157 is exterior to module 150, while clenching member is on the inside of module 150. As shown in FIG. 23, clenching member 158 of pin 156 holds a doubled-over or folded portion of restraint 154. The doubled-over portion of restraint 154 results in the restraint 154 being shorter in length, which holds spring 153 in a compressed state. Holding spring 153 in a compressed state helps prevent accidental triggering.

FIGS. 24 and 25 illustrate the removal process of the pin 156. First, as shown in FIG. 24, as handle 157 is pulled away from module 150, clenching member 158 of pin releases its grip on restraint 154. The doubled-over portion of restraint 154 begins to straighten out.

In FIG. 25, pin 156 is removed from module 150, and the restraint 154 is straight. When restraint 154 is straight, with no doubled-over portion, restraint 154 has an increased length, and spring 153 is free to move as it is no longer held in a compressed state by the shorter length restraint. FIG. 25 thus shows the triggerable position, as spring movement allows for needle guard 152 to move in relation to device 151.

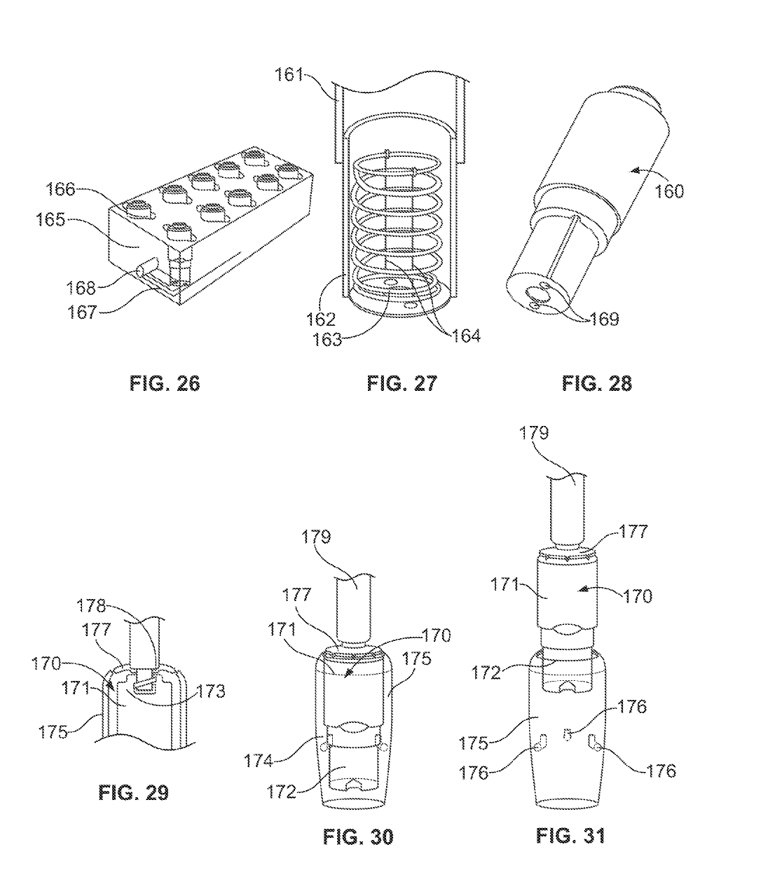

FIGS. 26-28 illustrate an exemplary alternative embodiment to the module with restraints of FIGS. 23-25. FIG. 26 illustrates a plurality of modules 160 within a packaging 165. Modules comprise a device 161 and a needle guard 162. Packaging 165 comprises a power source 165, a plurality of ports 166, and a plurality of electronic wires 167. A medicated module 160 is inserted into each of the ports 166. A first end of each electronic wire 167 is attached to power source 165, and the second end of each electronic wire 167 is attached to a medicated module residing in one of the ports 166. Preferably, two electronic wires 167 are attached to each module. Each of the second ends of the electronic wires 167 are attached to the bottom surface of needle guard 162.

As shown in FIG. 27, within needle guard 162 is a compression spring 163, a pair of fuse wires 164, and a pair of current transmitters 169. Each fuse wire 164 is attached at one end to current transmitter 169, and at the other end to a part of spring 163. Fuse wire 164 holds spring 163 in a compressed state, not allowing spring 163 to move a significant distance. The fuse wires 164 are thus positioned to reach across spring 163 axially, similar to the restraint 154 discussed with reference to FIGS. 23-25. Each fuse wire 164 may prevent spring 163 from moving at all. FIG. 28 illustrates the medicated module and shows the current transmitters 169 on the bottom surface of the needle guard 162.

Power is generated in the power source 165, and the power is transmitted as electric current through electric wires 167 to the fuse wires 164 within needle guard 162, via current transmitters 169. Fuse wires 164 receive enough current to melt. When fuse wires 164 melt, they no longer restrain compression spring 163 and spring 163 is free to relax and allow for elements such as a hub within medicated module 160 to move.

The fuse wires 164 may be made of zinc, copper, silver, aluminum, or alloys to provide stable and predictable characteristics. The fuse ideally would carry its rated current indefinitely, and melt quickly on a small excess. Alternatively, contact may be made (and the circuit completed) when the device is removed from the packaging, e.g., rotated to remove. One advantage of such a configuration is that it would help reduce electrical drain.

FIGS. 29-31 illustrate a module 170, such as the module 4 illustrated in FIGS. 2 and 7, and a secondary packaging 175. As shown in FIG. 30, module 170 comprises a device 171 and a needle guard 172. At the proximal end of device 171 is a hole 173. Device 171 also comprises a plurality of slots 174 at its distal end.

As shown in FIG. 30, secondary packaging 175 comprises a plurality of pegs 176 that extend from the interior surface of the packaging 175, a lid 177, and a lid 177. Lid 177 is detachable, and may comprise weakened areas along its perimeter. Lid 177 may be molded as part of secondary packaging 175.

FIG. 29 is a cross-sectional partial view of the module 170 within the secondary packaging 175. A primary device 179 has been inserted and punctured lid 177, creating a hole 178, and moving into device hole 173. Primary device 179 may comprise an attachment means such as a thread that screws into a corresponding groove within device 171. Primary device may be, for example, a drug delivery device such as the drug delivery device illustrated in FIG. 1.

FIG. 30 is a perspective view of the module 170 within secondary packaging 175. As shown in FIG. 30, primary device 179 is affixed to module 170. Pegs 176 are inserted into slots 174, restraining movement of the needle guard 172 relative to the device 171.

FIG. 31 is a perspective view of the module 170 being pulled out of secondary packaging 175. Module 170 is preferably removed by pulling primary device 179 once it is connected to medicated module 170 via the attachment means. Lid 177 remains attached to medicated module 170 and primary device 179 as the primary device is withdrawn from secondary packaging 175. Slots 174 are configured such that they can be pulled out of pegs 176 when the module 170 is pulled out of secondary packaging. This allows relative motion of the needle guard and the body, thus, leading to a triggerable state.

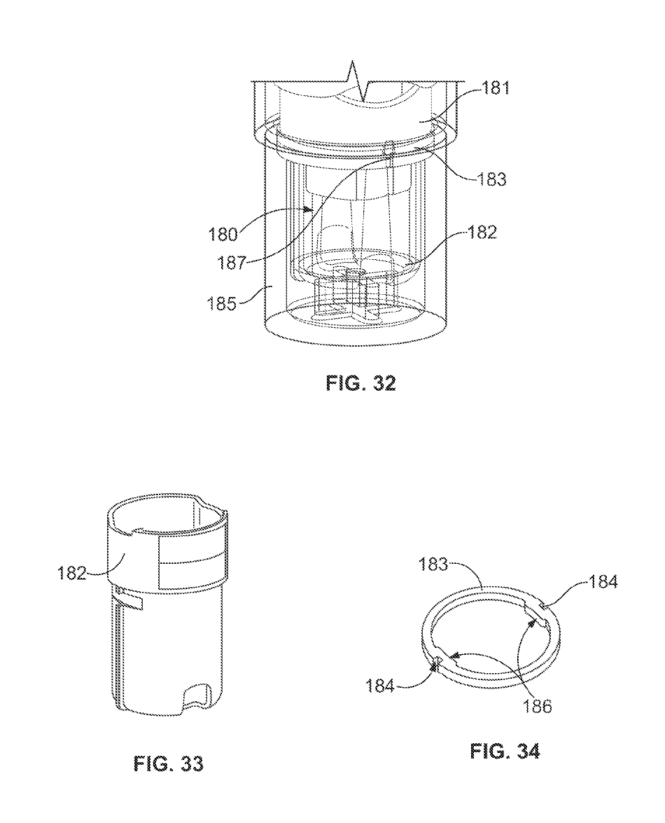

FIG. 32 is a cross-sectional partial view of a module 180, such as the medicated module 4 illustrated in FIGS. 2 and 7, within a secondary packaging 185. Medicated module 180 comprises a device 181, a needle guard 182, and a clip ring 183.

As shown in FIG. 34, clip ring 183 comprises a plurality of cut-outs 184 along the exterior circumference of the ring, and a plurality of lugs 186 along the inner circumference of the ring 183.

FIG. 33 shows the needle guard 182 of FIG. 32. As shown in FIG. 33, a groove 187 is present along at least part of the exterior surface circumference of needle guard 182.

To attach ring 183 to needle guard 182, lugs 186 engage with groove 187. Ring 183 may be assembled onto needle guard 182 during the manufacturing process. The plurality of cut-outs 184 on the exterior circumference or perimeter of ring 183 snap into secondary packaging 185. In one arrangement, this is an irreversible connection. That is, once the ring 183 is snapped into the secondary packaging 185 it cannot be removed. Therefore, the only possible action is to remove the secondary device 185 from the ring 183 where the force required to remove lugs 186 from groove 187 is less than the force required to remove cut-outs 184 from the notch in the secondary packaging.

While the module 180 is within secondary packaging 185, ring 183, which extends beyond the perimeter of needle guard 182, jams against device body 181, preventing significant movement of the needle guard 182 with respect to device body 181. This is the trigger locked position.

When a user removes the module 180 from secondary packaging 185 prior to use, ring 183 remains attached to packaging 185. With ring 183 removed, the module 180 is active because the needle guard 182 can move in relation to device 181, and is in the triggerable position.

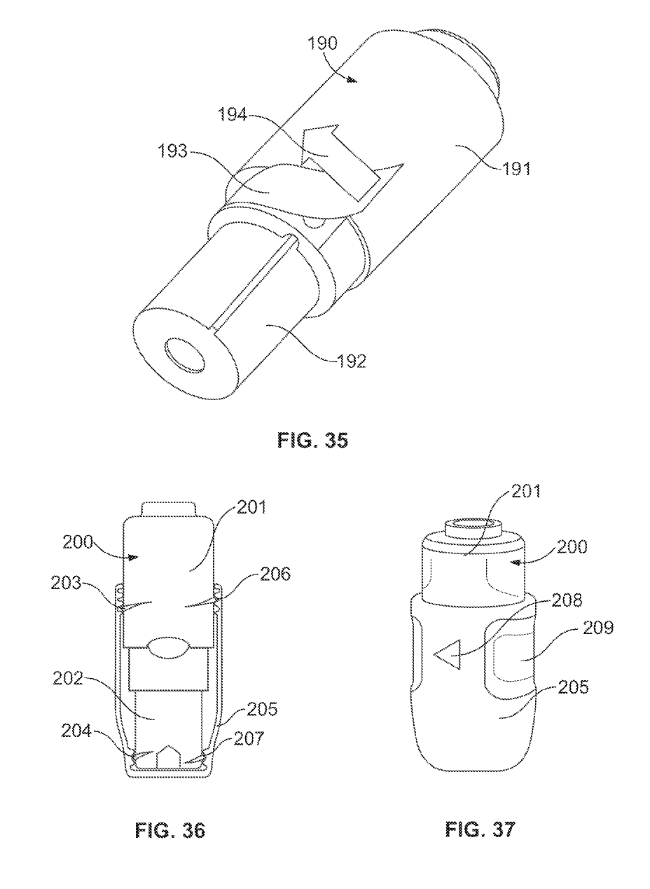

FIG. 35 is a perspective view of a module 190, such as the medicated module 4 illustrated in FIGS. 2 and 7. Module 190 comprises a device body 191, a needle guard 192, and a tear or peel-off strip 193. Peel-off strip 193 is applied to device body 191, and includes a feature that prevents movement of needle guard 192 relative to device body 191. For example, the inside surface of the strip 193 could comprise a peg, a pin, a slider element or the like. These features could fit within a corresponding orifice within both device body 191 and needle guard 192, to prevent the two components from moving relative to one another, thereby prevent accidental triggering.

A user can peel off the strip 193, in the direction shown with arrow 194. In an alternative embodiment, the user can tear the strip 193 if a shrink-wrapped or a semi-rigid tamper strip is used. Removal of strip 193 removes the blocking feature and allows the module to be activated.

FIG. 36 is a cross-sectional view of a module, 200 such as the medicated module 4 illustrated in FIGS. 2 and 7, within a secondary packaging 205. Module 200 comprises a device body 201 and a needle guard 202. Device body 201 comprises a thread 203 along the exterior circumference of the device. Needle guard 202 comprises a thread 204 along the exterior circumference of the device. Device body thread 203 may have a different pitch than needle guard thread 204. This difference in pitch may act to pre-load components in tension. For example, by winding one part into the secondary packaging slightly faster than the other part, the two components may be effectively ‘jacked’ apart and pulled apart from each other. This can be done because they are engaged with the same component—the secondary packaging. If the difference in pitch is too great, the threads will lock up before the device can be fully wound into the packaging.

Secondary packaging comprises a first threaded groove 206 that corresponds to device thread 203, and a second threaded groove 207 that corresponds to needle guard thread 204.

When module 200 is placed within secondary packaging 205, the grooves are aligned with their corresponding threads, and secondary packaging 205 is turned or rotated until the threads have sufficiently traveled the length of the grooves, such that medicated module 200 is firmly secure within secondary packaging 205. This is the trigger locked position, the module is threadedly engaged with both sets of threads and the threads may be shallow enough so as not to overhaul under axial load.

FIG. 37 is a perspective view of the module 200 within secondary packaging 205 of FIG. 36. An arrow 208 may be printed on the exterior surface of secondary packaging 205, to show a user the direction the user should twist the packaging 205 to remove packaging 205 from module 200. Grips 209 may also be present on the exterior surface of secondary packaging 205.

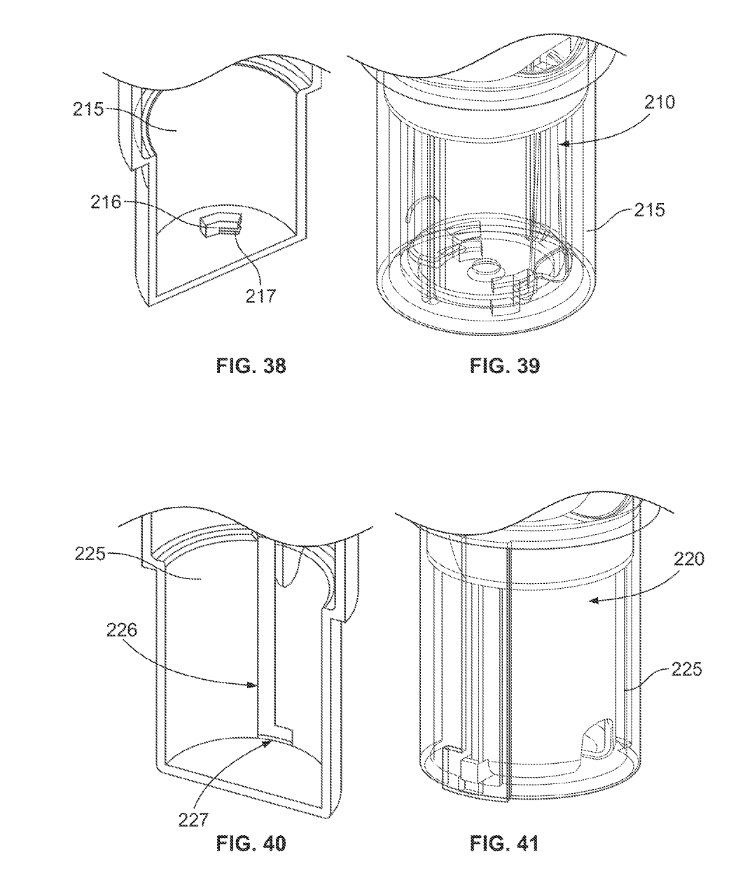

FIG. 38 is a partial, cross-sectional view of a secondary packaging 215. At the interior, bottom surface of secondary packaging 215 is a raised lug 216 with a moulding shut-off 217. Raised lug 216 is positioned on the bottom surface such that a module 200 with a corresponding aperture in the distal face of needle guard, when placed within secondary packaging 215 (as shown in the cross-sectional view of FIG. 39) can be fitted axially by having the aperture fit over lug 216. Once the aperture of the medicated module 210 is placed over lug 216, the module 210 is rotated into place over lug 216. This engages the aperture with lug 216 and ensures that the needle guard of the module 200 cannot move relative to the device portion of the module 210.

FIG. 40 is a partial, cross-sectional view of an alternative secondary packaging 225. At the interior wall surface of secondary packaging 225 is a groove 226 with a bayonet pocket 227. Groove 226 is positioned on the wall surface such that a medicated module 220 with a corresponding indent lug on needle guard, when placed within secondary packaging 225 (as shown in the cross-sectional view of FIG. 41), can rotate into place by having the lug pass into the groove 226. Once medicated module 220 has slid axially down into place through 226, the needle guard of the module 220 is rotated to engage lug in bayonet pocket 227. In this state, needle guard 220 cannot move relative to the device portion of the module 200 because the body of the device is sat on a ledge in the secondary packaging. This ledge stops the body from moving down onto the guard while the lug/pocket arrangement stops the needle guard moving up into the body. These are the two scenarios that would lead to relative motion and triggering. Bayonet pocket 227 prevents axial movement of the needle guard of module 220 within packaging 225.

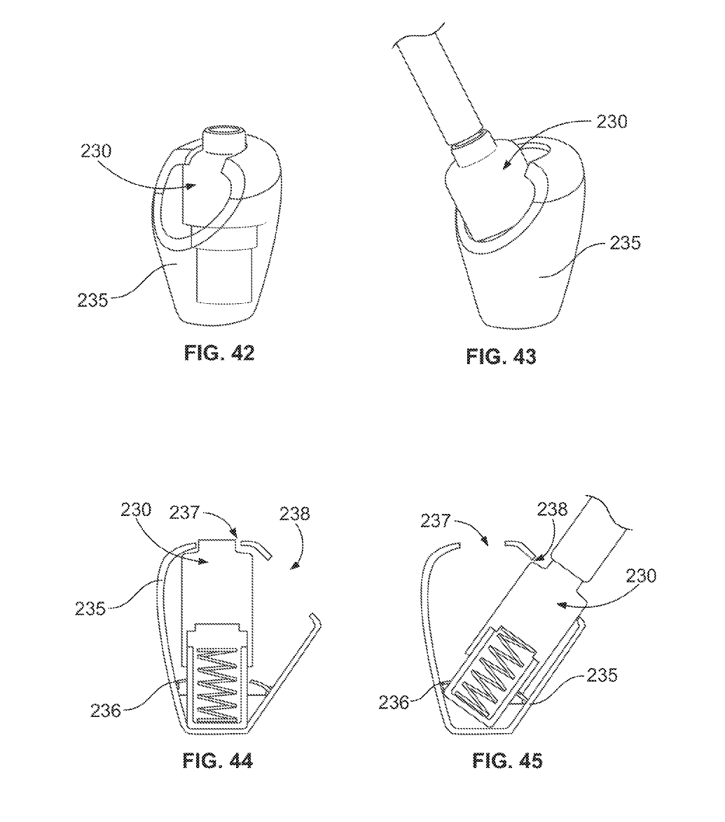

FIG. 42 is a perspective view of an exemplary embodiment of a module 230 within a secondary packaging 235. Secondary packaging comprises a first opening 237 and a second opening 238. FIG. 43 is a perspective view of the module 230 of FIG. 42 being removed from secondary packaging 235.

FIG. 44 is a cross-sectional view of the medicated module within the secondary packaging 235 in the position shown in FIG. 42.

To remove module 230 from secondary packaging 235, module 230 is first pushed down or compressed in the direction shown by arrow 238 in FIG. 45, such that the tip 239 of module 230 is below the edges defining first opening 237. Once sufficiently compressed, module 230 can be slanted so that the module is aligned with second opening 238. The compression force is then released, and module 230 extends through second opening 238. Module 230 can now be removed from its position in FIG. 45, and used in a triggerable position. The biasing member is over-extended in the stored state such that it can absorb any impact/shock during transit etc. without travelling far enough to trigger the device. Essentially, in this arrangement, the biasing member is used as a type of shock absorber but requires enlargement of the device to accommodate the additional spring extension.

In any of the above described embodiments, preferably the medicated module is provided by a drug manufacturer as a stand-alone and separate device that is sealed to preserve sterility. The sterile seal of the module is preferably designed to be opened automatically, e.g. by cutting, tearing or peeling, when the medicated module is advanced or attached to the primary drug delivery device by the user. Features such as angled surfaces on the end of the injection device or features inside the module may assist this opening of the seal.

The module of our invention should be designed to operate in conjunction with a multiple use injection device, preferably a pen-type multi-dose injection device, similar to what is illustrated in FIG. 1. The injection device could be a reusable or disposable device. By disposable device it is meant an injection device that is obtained from the manufacturer preloaded with medicament and cannot be reloaded with new medicament after the initial medicament is exhausted. The device may be a fixed dose or a settable dose and preferably a multi-dose device, however, in some cases it may be beneficial to use a single dose, disposable device.

A typical injection device contains a cartridge or other reservoir of primary medication. This cartridge is typically cylindrical in shape and is usually manufactured in glass. The cartridge is sealed at one end with a rubber bung and at the other end by a rubber septum. The injection device is designed to deliver multiple injections. The delivery mechanism is typically powered by a manual action of the user, however, the injection mechanism may also be powered by other means such as a spring, compressed fluid or electrical energy. In a preferred embodiment, the delivery mechanism comprises a spindle that engages a piston in the reservoir. In a further embodiment the spindle is a rotatable piston rod comprising two distinct threads.

Exemplary embodiments of the present invention have been described. Those skilled in the art will understand, however, that changes and modifications may be made to these embodiments without departing from the true scope and spirit of the present invention, which is defined by the claims.

1-13. (canceled)

14. A module attachable to a drug delivery device, comprising, a device having an inner surface, a proximal end and a distal end, where the proximal end has an upper hub holding a needle cannula and a connector configured for attachment to a drug delivery device;
a housing having an outer surface and slidably engaged with an upper radial stand off on the inner surface of the housing;
a needle guard;
a lower hub slidably engaged with the outer surface of the housing and slidably engaged with the inner surface of the needle guard; and
at least one opening through both the needle guard and the device for the insertion of a restraining element, wherein the inserted restraining element prevents the needle guard from moving relative to the device, wherein the restraining element is a peg located on a secondary packaging that contains the module.

15. The module of claim 14 wherein the restraining element is re-usable.

16. A module attachable to a drug delivery device, comprising,
a device having an inner surface, a proximal end and a distal end, where the proximal end has an upper hub holding a first double-ended needle cannula and a connector configured for attachment to a drug delivery device;
a housing having an outer surface and slidably engaged with an upper radial stand off on the inner surface of the housing;
a needle guard with at least one female component and a biasing member;
a lower hub slidably engaged with the outer surface of the housing and slidably engaged with the inner surface of the needle guard; and
a restraining element, wherein the restraining element mates with the female component to prevent the needle guard from moving relative to the device.

17. The module of claim 16 wherein the female component is an indent, and the restraining element is a sprung latch, a portion of which fits within indent.

18. The module of claim 16 further comprising a pin that is removably insertable into the female component, and a restraining wire that axially spans at least a portion of the biasing member, wherein the pin shortens the length of the restraining wire, thereby compressing the biasing member.

19. A module attachable to a drug delivery device, comprising,
a secondary packaging that conforms to the exterior of the module so as to restrain elements of the module from moving relative to each other.

20. A module attachable to a drug delivery device, comprising,
a device body; and
a needle guard with at least one restraining element;
wherein the restraining element interacts with a secondary packaging to restrain the device body from moving relative to the needle guard.

21. The module of claim 20 wherein the restraining element is a fuse wire and wherein the secondary packaging sends current through the fuse wire.

22. The module of claim 20 wherein the restraining element is a ring that attaches to the exterior of the needle guard.

23. The module of claim 20 wherein the restraining element is a thread on an exterior surface of the needle guard that mates with a thread in the secondary packaging.

24. The module of claim 14 further comprising a reservoir within the housing comprising a single dose of a medicament.

25. An assembly, comprising a module according to claim 14 and a cover arranged to cover at least a portion of the module, the module having an initial and an actuated state, the restraining element of the module having a first position, preventing movement of the needle guard and a second position allowing movement of the needle guard, wherein movement of at least a portion of the cover relative to the module brings the restraining element from the first position to the second position thereby changing state of the module from the initial state to the actuated state.

26. An assembly as in claim 25, wherein the cover is in the form of a container comprising:
a cavity portion and a closure member attached thereto and which together form an enclosure in which the module is arranged initially,
wherein movement of the closure member relative to the module brings the restraining element from the first position to the second position thereby changing state of the module from the initial state to the actuated state.
